(19) United States
(12) Patent Application Publication
(10) Pub. No.: US 2025/0304857 A1
(43) Pub. Date: Oct. 2, 2025

(54) AROMATIC AMINE COMPOUND, LIQUID CRYSTAL COMPOSITION, LIQUID CRYSTAL ELEMENT, DISPLAY APPARATUS, AND LIGHT CONTROL APPARATUS

(71) Applicant: Murata Manufacturing Co., Ltd., Nagaokakyo-shi (JP)

(72) Inventor: Masashi UEBE, Nagaokakyo-shi (JP)

(21) Appl. No.: 19/179,508

(22) Filed: Apr. 15, 2025

Related U.S. Application Data
(63) Continuation of application No. PCT/JP2023/039232, filed on Oct. 31, 2023.

(30) Foreign Application Priority Data
Nov. 4, 2022 (JP) 2022-177592

(51) Int. Cl. C09K 19/58; C07D 321/00

(52) U.S. Cl. CPC C09K 19/588; C07D 321/00

(57) ABSTRACT

An aromatic amine compound is represented by:

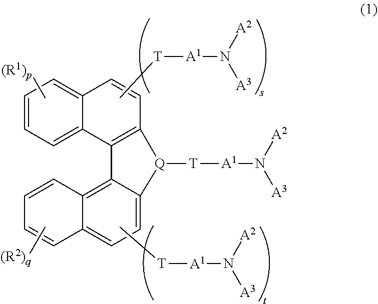

A¹s each independently represent a substituted or unsubstituted alkylene group or a substituted or unsubstituted divalent aromatic group, and A²s and A³s each independently represent a substituted or unsubstituted alkyl group or a substituted or unsubstituted aromatic group, where at least one of A¹, A² and A³ represents an aromatic group. s and t each independently represent an integer from 0 to 6. R¹ and R² each independently represent a substituent. p+s and q+t each independently represent an integer from 0 to 6. Ts each independently represent a divalent linking group formed from at least one selected from a carbonyl group, an oxygen atom, an imino group, and an alkylene group. Q represents a trivalent linking group composed of at least one selected from an oxygen atom, a nitrogen atom, a carbon atom, a phosphorus atom, a sulfur atom, and a hydrogen atom.

### CROSS REFERENCE TO RELATED APPLICATIONS

The present application is a continuation of International application No. PCT/JP2023/039232, filed Oct. 31, 2023, which claims priority to Japanese Patent Application No. 2022-177592, filed Nov. 4, 2022, the entire contents of each of which are incorporated herein by reference.

### TECHNICAL FIELD

The present disclosure relates to an aromatic amine compound, a liquid crystal composition, a liquid crystal element, a display apparatus, and a light control apparatus.

### BACKGROUND ART

Liquid crystal display devices are used not only in personal computers and television sets but also in a variety of other places. The liquid crystal display device has a backlight, which is the key to the further reduction in power consumption of the device. Cholesteric liquid crystals can selectively reflect light, and a reflective display having the cholesteric liquid crystals consumes less power to control light. For example, Japanese Unexamined Patent Application Publication No. 2019-151597 and J. Am. Chem. Soc., 2018, 140, 10946. propose that a compound having a binaphthyl skeleton which serves as a chiral moiety and ferrocene which is introduced to the binaphthyl skeleton to serve as a redox site be employed as a chiral dopant that forms cholesteric liquid crystals. The documents further indicate that, regarding a liquid crystal composition layer containing the chiral dopant having ferrocene introduced thereto, the reflection wavelength of the cholesteric liquid crystals can be controlled through redox reactions induced by voltage application.

### SUMMARY OF THE DISCLOSURE

An object of one aspect of the present disclosure is to provide an aromatic amine compound that can serve as a chiral dopant that is stably oxidizable and reducible in a liquid crystal composition.

### Solution to Problem

A first aspect is an aromatic amine compound represented by formula (1) below.

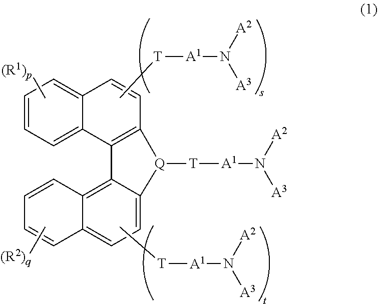

In the formula (1), A¹s each independently represent a substituted or unsubstituted alkylene group or a substituted or unsubstituted divalent aromatic group. A²s and A³s each independently represent a substituted or unsubstituted alkyl group or a substituted or unsubstituted aromatic group. At least one of A¹, A² and A³ represents an aromatic group. s and t each independently represent an integer from 0 to 6. R¹ and R² each independently represent a substituent. p+s and q+t each independently represent an integer from 0 to 6. Ts each independently represent a divalent linking group formed from at least one kind selected from a carbonyl group, an oxygen atom, an imino group, and an alkylene group. Q represents a trivalent linking group composed of at least one kind selected from an oxygen atom, a nitrogen atom, a carbon atom, a phosphorus atom, a sulfur atom, and a hydrogen atom.

A second aspect is a liquid crystal composition containing the aromatic amine compound of the first aspect. A third aspect is a liquid crystal element including a liquid crystal layer containing the liquid crystal composition of the second aspect and a pair of electrodes configured to apply voltage to the liquid crystal layer. A fourth aspect is a display apparatus or light control apparatus including the liquid crystal element of the third aspect.

According to one aspect of the present disclosure, an aromatic amine compound that can serve as a chiral dopant that is stably oxidizable and reducible in a liquid crystal composition can be provided.

### BRIEF DESCRIPTION OF THE DRAWINGS

FIG. 1(a) is an example of a cyclic voltammogram of a compound according to Example 3 in which ferrocene is used as a standard, and FIG. 1(b) is an example of a cyclic voltammogram of a compound according to Comparative Example 1 in which ferrocene is used as a standard.

FIG. 2(a) is an example of an absorption spectrum of the compound according to Example 3, and FIG. 2(b) is an example of an absorption spectrum of the compound according to Comparative Example 1.

FIG. 3 is an example of transmission spectra of liquid crystal compositions containing compounds according to Examples and Comparative Example.

FIG. 4(a) is an example of a transmission spectrum before application of a direct current voltage, and FIG. 4(b) is an example of a transmission spectrum after application of a direct current voltage.

### DESCRIPTION OF THE PREFERRED EMBODIMENTS

The term “step” used herein encompasses not only an independent step, but also a step not clearly distinguished from another step as long as an intended purpose of the step is accomplished. In addition, the content of each component of a composition means, in the case where multiple substances corresponding to the component are present in the composition, the total amount of the multiple substances that are present in the composition, unless otherwise specified. Furthermore, the upper limit and the lower limit of a numerical range described herein can be appropriately selected from numerical values exemplified in relation to the numerical range, and combined. An embodiment of the present disclosure will be described in detail below. However, the embodiment described below is merely an example of the aromatic amine compound, the liquid crystal composition, the liquid crystal element, the display apparatus, or the light control apparatus for the purpose of embodying technical thoughts of the present disclosure, and the present disclosure is not limited to the aromatic amine compound, the liquid crystal composition, the liquid crystal element, the display apparatus, or the light control apparatus described below.

### Aromatic Amine Compound

The aromatic amine compound is represented by formula (1) below. The aromatic amine compound includes, as represented by the formula (1) below, a binaphthyl skeleton serving as a chiral moiety, and an aromatic amine skeleton serving as a redox site. Due to the aromatic amine skeleton serving as a redox site, the compound represented by the formula (1) below can stably and electrochemically repeat redox reactions under the atmosphere, in a solution, in a liquid crystal composition, or the like. That is, the aromatic amine compound represented by the formula (1) can reversibly develop ionicity and non-ionicity in response to electrical stimuli. It is considered that such a compound that is optically active and responsive to electrical stimuli can, in cholesteric liquid crystals, for example, control the molecular arrangement of the helical structure of the cholesteric liquid crystals through electrical stimuli. This allows the period (pitch) of the helical structure formed by cholesteric liquid crystals to be controlled, and accordingly, the wavelength of circularly-polarized light to be selectively reflected by the cholesteric liquid crystals can be controlled. Specifically, light having a long wavelength can be reflected when the helical structure has a long pitch, whereas light having a short wavelength can be reflected when the pitch is short.

The aromatic amine compound may have no absorption in the visible light range. This allows a colorless liquid crystal composition having no absorption in the visible light range to be produced, for example. The aromatic amine compound having no absorption in the visible light range can be obtained by, for example, appropriately selecting a substituent on the aromatic amine skeleton, a substituent on the binaphthyl skeleton, or the like.

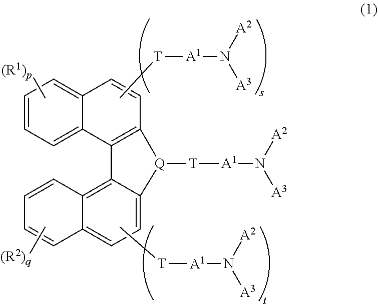

In the formula (1), A¹s each independently represent a substituted or unsubstituted alkylene group or a substituted or unsubstituted divalent aromatic group. A²s and A³s each independently represent a substituted or unsubstituted alkyl group or a substituted or unsubstituted aromatic group. At least one of A¹, A² and A³ represents an aromatic group.

An alkylene group represented by A¹ may have a linear, branched, or annular form, or a combination thereof. The number of carbon atoms of the alkylene group represented by A¹ may be, for example, 1 to 20, preferably 1 or more, or 10 or less. A divalent aromatic group represented by A¹ is formed by removing two hydrogen atoms from an aromatic hydrocarbon compound or aromatic heterocyclic compound. The aromatic hydrocarbon compound may have 6 to 18 carbon atoms, preferably 6 carbon atoms. The aromatic hydrocarbon compound may contain at least one kind selected from the group consisting of benzene, naphthalene, and anthracene. The aromatic heterocyclic compound may contain, as a heteroatom, at least one kind selected from the group consisting of a nitrogen atom, an oxygen atom, and a sulfur atom. The number of members of the aromatic heterocyclic compound may be, for example, 5 to 10, preferably 6 or less. The aromatic heterocyclic compound may contain at least one kind selected from the group consisting of pyridine, furan, and thiophene. In the case where a plurality of alkylene groups or divalent aromatic groups represented by A¹ are present in the aromatic amine compound, they may the same or different.

The alkylene group or divalent aromatic group represented by A¹ may have a substituent. The substituent on A¹ may be at least one kind of substituent selected from the group consisting of a substituted or unsubstituted hydrocarbon group, a nitro group, a cyano group, a halogen atom, a hydroxy group, an alkoxy group, an acyl group, an alkoxycarbonyl group, a carboxy group, an aliphatic amino group, and an aromatic amino group.

The hydrocarbon group as the substituent may be an aliphatic group or an aromatic group. The aliphatic group may be a saturated aliphatic group or an unsaturated aliphatic group. The aliphatic group may have a linear, branched, or annular form, or a combination thereof. The aliphatic group may have, for example, 1 to 20 carbon atoms, preferably 1 to 10 carbon atoms, or 1 to 6 carbon atoms. Examples of a substituent on the aliphatic group include a halogen atom, an aryl group, an alkoxy group, and the like. The aromatic group may have, for example, 6 to 18 carbon atoms, preferably 6 carbon atoms. Examples of a substituent on the aromatic group include a halogen atom, an aliphatic group having 1 to 20 carbon atoms, an alkoxy group, an acyl group, an alkoxycarbonyl group, and the like.

The halogen atom as the substituent may include a fluorine atom, a chlorine atom, a bromine atom, and the like. The alkoxy group as the substituent may have an aliphatic group having 1 to 20 carbon atoms, preferably an aliphatic group having 1 to 10 carbon atoms. The acyl group as the substituent may have an aliphatic group having 1 to 20 carbon atoms, preferably an aliphatic group having 1 to 6 carbon atoms. The alkoxycarbonyl group as the substituent may have an aliphatic group having 1 to 20 carbon atoms, preferably an aliphatic group having 1 to 6 carbon atoms.

An aliphatic group of the aliphatic amino group as the substituent may be a saturated aliphatic group or an unsaturated aliphatic group. The aliphatic group may have a linear, branched, or annular form, or a combination thereof. The aliphatic group may have, for example, 1 to 20 carbon atoms, preferably 1 to 10 carbon atoms, or 1 to 6 carbon atoms. The aliphatic amino group may be a monosubstituted aliphatic amino group having one aliphatic group, or a disubstituted aliphatic amino group having two aliphatic groups. The aliphatic amino group may further have a substituent on the moiety of the aliphatic group. Examples of the substituent on the aliphatic group include a halogen atom, an aryl group, an alkoxy group, an alkylamino group, an arylamino group, and the like. The number of substitutions in the aliphatic group may be, for example, 0 to 20, preferably 10 or less.

An aromatic group of the aromatic amino group as the substituent may be an aromatic hydrocarbon group, or an aromatic heterocyclic ring group. The aromatic hydrocarbon group may have, for example, 6 to 18 carbon atoms, preferably 6 to 12 carbon atoms. The aromatic hydrocarbon group may include at least one kind selected from the group consisting of a phenyl group, a naphthyl group, and an anthracenyl group.

The aromatic heterocyclic ring group may include, as a heteroatom, at least one kind selected from the group consisting of a nitrogen atom, an oxygen atom, and a sulfur atom. The number of members of the aromatic heterocyclic ring group may be, for example, 5 to 10, preferably 6 or less. The aromatic heterocyclic ring group may include at least one kind selected from the group consisting of a pyridyl group, a furyl group, and a thienyl group. The aromatic amino group may be a monosubstituted aromatic amino group having one aromatic group, or a disubstituted aromatic amino group having two aromatic groups. The aromatic amino group may further have a substituent on the moiety of the aromatic group. Examples of the substituent on the aromatic group include a halogen atom, an aryl group, an alkoxy group, an alkylamino group, an arylamino group, an alkyl group, and the like. The number of substitutions in the aromatic group may be, for example, 0 to 8, preferably 5 or less.

The number of substitutions in the alkylene group or divalent aromatic group represented by A¹ may be, for example, 0 to 20, preferably 4 or less.

The alkyl group represented by A² or A³ may have a linear, branched, or annular form, or a combination thereof. The alkyl group represented by A² or A³ may have, for example, 1 to 20 carbon atoms inclusive, preferably 1 or more carbon atoms, or 6 or less carbon atoms. The aromatic group represented by A² or A³ is formed by removing one hydrogen atom from an aromatic hydrocarbon compound or an aromatic heterocyclic compound. Details of the aromatic hydrocarbon compound and the aromatic heterocyclic compound are similar to those of the aromatic hydrocarbon compound and the aromatic heterocyclic compound for A¹. In the case where a plurality of alkyl groups or aromatic groups represented by A² or A³ are present in the aromatic amine compound, they may the same or different.

The alkyl group or aromatic group represented by A² or A³ may have a substituent. Examples of the substituent on A² or A³ are similar to those of the substituent on A¹. The number of substitutions in the alkyl group or aromatic group represented by A² or A³ may be, for example, 0 to 20, preferably 5 or less.

At least one of the aromatic group represented by A² or the aromatic group represented by A³ may have a substituent, and may have an aromatic amino group as the substituent. The aromatic amino group with which the aromatic group represented by A² or A³ is substituted may be a disubstituted aromatic amino group, and the aromatic group of the aromatic amino group may further have a substituent. Examples of the substituent on the aromatic group include a halogen atom, an aryl group, an alkoxy group, an alkylamino group, an arylamino group, an alkyl group, and the like. The number of substitutions in the aromatic group may be, for example, 0 to 9, preferably 1 to 5.

At least one of A¹, A² and A³ represents an aromatic group, but preferably at least two of them may be an aromatic group, and more preferably three of them may be an aromatic group. Furthermore, out of A¹, A² and A³, at least A¹ may be an aromatic group, and at least one of A² and A³ may be an aromatic group.

s and t each independently represent an integer from 0 to 6, preferably may be an integer equal to or less than 5 or an integer equal to or less than 2, and may be an integer equal to or more than 1. In addition, p and q may each independently represent an integer from 0 to 6, preferably may be an integer equal to or less than 5 or an integer equal to or less than 2, and may be an integer equal to or more than 1. Furthermore, pts and q+t each independently represent an integer from 0 to 6, preferably may be an integer equal to or less than 5 or an integer equal to or less than 2, and may be an integer equal to or more than 1.

R¹ and R² each independently represent a substituent. Examples of the substituent represented by R¹ or R² are similar to those of the substituent on A¹. In the case where a plurality of substituents represented by R¹ or R² are present in the aromatic amine compound, they may the same or different.

Ts each independently represent a divalent linking group formed from at least one kind selected from the group consisting of a carbonyl group, an oxygen atom, an imino group, and an alkylene group. The imino group as T may be substituted with a hydrocarbon group. Examples of the hydrocarbon group with which the imino group is substituted are similar to those of the hydrocarbon group as the substituent on A¹. The alkylene group as T may have a linear, branched, or annular form, or a combination thereof. The alkylene group as T may have, for example, 1 to 20 carbon atoms inclusive, preferably 10 or less carbon atoms, or 6 or less carbon atoms. The divalent linking group represented by T may be a carbonyl group, an oxygen atom, an imino group or an alkylene group and, for example, may include an ester bond formed by bonding of a carbonyl group and an oxygen atom, may include an amido bond, a urea bond, a urethane bond, or the like formed by bonding of a carbonyl group and an imino group, and may include an ether bond formed by bonding of an oxygen atom and an alkylene group. In the case where a plurality of divalent linking groups represented by T are present in the aromatic amine compound, they may the same or different.

The divalent linking group represented by T may include at least a carbonyl group. Specific examples of the divalent linking group represented by T include a carbonyl group, an oxygen atom, an imino group, an alkylene group, a carbonyloxy group, an oxycarbonyl group, an alkylenecarbonyloxy group, an alkyleneoxycarbonyl group, a carbonyloxyalkylene group, an oxycarbonylalkylene group, an iminocarbonyl group, an alkyleneiminocarbonyl group, a carbonylimino group, a carbonyliminoalkylene group, an alkyleneoxy group, an oxyalkylene group, an iminocarbonylimino group, an oxycarbonylimino group, an iminocarbonyloxy group, and the like. Preferable examples of the divalent linking group represented by T include a carbonyloxy group, an oxycarbonyl group, an oxygen atom, and the like.

Q represents a trivalent linking group composed of at least one kind selected from the group consisting of an oxygen atom, a nitrogen atom, a carbon atom, a phosphorus atom, a sulfur atom, and a hydrogen atom. Q may be a trivalent linking group represented by formula (2a) or (2b) below, for example.

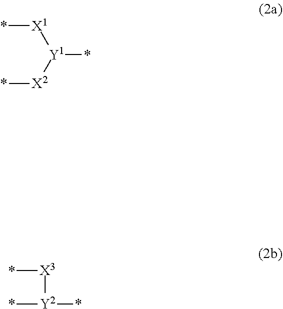

In the formulas (2a) and (2b), * denotes a site of bonding to another atom. X¹, X² and X³ each independently include at least one kind selected from the group consisting of an oxygen atom, a sulfur atom, —C(R³)(R⁴)—, and —N(R⁵)—. R³, R⁴ and R⁵ each independently represent a hydrogen atom, a substituted or unsubstituted alkyl group, or a substituted or unsubstituted aromatic group. Y¹ and Y² each independently represent at least one kind selected from the group consisting of a substituted or unsubstituted alkanetriyl group, a nitrogen atom, and —P(═O)(O—)—.

The alkyl group represented by R³, R⁴ or R⁵ may have a linear, branched, or annular form, or a combination thereof. The alkyl group represented by R³, R⁴ or R⁵ may have, for example, 1 to 20 carbon atoms inclusive, preferably 6 or less carbon atoms. The aromatic group represented by R³, R⁴ or R⁵ is formed by removing one hydrogen atom from an aromatic hydrocarbon compound or an aromatic heterocyclic compound. The aromatic hydrocarbon compound and the aromatic heterocyclic compound are as described above. Examples of the substituent on R³, R⁴ or R⁵ are similar to those of the substituent on A¹.

The alkanetriyl group represented by Y¹ or Y² is formed by removing three hydrogen atoms from an alkane. The alkane from which the alkanetriyl group is to be formed may have, for example, 1 to 20 carbon atoms inclusive, preferably 6 or less carbon atoms. Examples of the substituent on Y¹ or Y² are similar to those of the substituent on A¹.

Although specific examples of the trivalent linking group represented by the formula (2a) include the following linking groups, the present disclosure is not limited thereto. Note that, as to the trivalent linking group represented by the formula (2a), X¹ and X² may be bonded to the binaphthyl moiety in the formula (1), and Y¹ may be bonded to T in the formula (1).

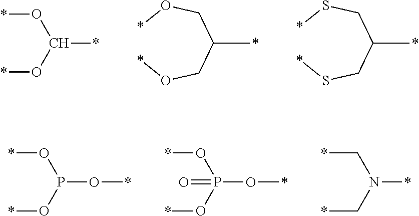 [SMILES: *OC(*)O* ; *OCC(*)CO* ; *SCC(*)CS* ; *OP(O*)O* ; *OP(=O)(O*)O* ; *CN(*)C*]

Although specific examples of the trivalent linking group represented by the formula (2b) include the following linking groups, the present disclosure is not limited thereto. Note that, as to the trivalent linking group represented by the formula (2b), X³ and Y² may be bonded to the binaphthyl moiety in the formula (1), and Y² may be bonded to T in the formula (1).

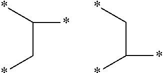

The trivalent linking group represented by Q may be preferably represented by the formula (2a), and more preferably, X¹ and X² in the formula (2a) may be an oxygen atom, and Y¹ may be a propane-1, 2, 3-triyl group.

The aromatic amine compound represented by the formula (1) can be produced in the following manner, for example. A dihaloalkane having a substituent is reacted with 1,1′-bi (2-naphthol) to introduce the trivalent linking group represented by Q, and an aromatic amine derivative is linked to the trivalent linking group represented by Q through a condensation reaction, a substitution reaction, a coupling reaction or the like, thereby producing the compound represented by the formula (1). Alternatively, by employing 1,1′-bi (2-naphthol) having an appropriate substituent on its naphthyl ring, the aromatic amine derivative can be linked on the naphthyl ring.

### Liquid Crystal Composition

The liquid crystal composition contains at least one kind of aromatic amine compound represented by the above formula (1). The liquid crystal composition containing the aromatic amine compound represented by the formula (1) can develop cholesteric liquid crystals, for example. In addition, the liquid crystal composition shows selective reflection, and can change the selectively reflected wavelength through redox reactions induced by an electric field. The content of the compound represented by the formula (1) in the liquid crystal composition may be, for example, 0.1 mol % to 10 mol %, preferably 0.5 mol % to 5 mol %.

The liquid crystal composition may contain the aromatic amine compound represented by the above formula (1) as a liquid crystal compound, and may contain a liquid crystal compound other than the aromatic amine compound represented by the above formula (1) as host liquid crystals, and contain the aromatic amine compound represented by the formula (1) as a chiral dopant.

Examples of the liquid crystal compound that constitutes the liquid crystal composition include a liquid crystal compound that shows a nematic phase and a liquid crystal compound that shows a smectic phase, and the liquid crystal compound that shows a nematic phase is preferable. Specific examples of the liquid crystal compound include an azomethine compound, a cyanobiphenyl compound, a cyanophenyl ester compound, a fluorine-substituted phenyl ester compound, a cyclohexane carboxylic acid phenyl ester compound, a fluorine-substituted cyclohexane carboxylic acid phenyl ester compound, a cyanophenylcyclohexane compound, a fluorine-substituted phenylcyclohexane compound, a cyanophenylpyrimidine compound, a fluorine-substituted phenylpyrimidine compound, an alkoxyphenylpyrimidine compound, a fluorine-substituted alkoxyphenylpyrimidine compound, a phenyldioxane compound, a tolan compound, a fluorine-substituted tolan compound, and an alkenylcyclohexylbenzonitrile compound. As to the details of the liquid crystal compound, for example, description in pp. 154-192 and pp. 715-722 of Handbook of Liquid Crystal Devices (Ekisyo Debaisu Handobukku) edited by the 142nd committee of Japan Society for the Promotion of Science, published by Nikkan Kogyo Shimbun, Ltd. in 1989 can be referred to.

The liquid crystal composition may further contain an electrolyte. The liquid crystal composition containing the electrolyte can have conductivity, which facilitates the redox reactions of the compound represented by the formula (1). The electrolyte may be a supporting electrolyte constituting the liquid crystal composition, and may be selected from compounds being highly soluble in the host liquid crystals. Examples of the electrolyte include a supporting electrolyte (for example, nBu₄NPF₆, nBu₄NBF₄, nBu₄NClO₄, or the like) generally used in electrochemistry, and an ionic liquid. Examples of the ionic liquid include 1-ethyl-3-methylimidazolium triflate, 1-ethyl-3-methylimidazolium hexafluorophosphate, and the like. The liquid crystal composition may contain one kind of electrolyte, or a combination of two or more electrolytes. The content of the electrolyte in the liquid crystal composition may be, for example, 0.1 mol % to 30 mol %, preferably 0.5 mol % to 15 mol %.

A variety of liquid crystal or non-liquid crystal compounds can be added to the liquid crystal composition for the purpose of, for example, changing the property of the host liquid crystals (for example, the temperature range of the liquid crystal phase) to a desired range, or facilitating redox reactions. In addition, the liquid crystal composition may contain an additive such as an ultraviolet absorber, an antioxidant, or the like. Furthermore, the liquid crystal composition may contain a chiral dopant other than the aromatic amine compound represented by the formula (1).

### Liquid Crystal Element

The liquid crystal element includes a liquid crystal layer containing the above-described liquid crystal composition, and a pair of electrodes for applying voltage to the liquid crystal layer. Owing to the liquid crystal layer containing the liquid crystal composition, the liquid crystal element can show a reflected color due to the development of cholesteric liquid crystals, for example. In addition, the reflected color can be changed through voltage application to the liquid crystal layer from the pair of electrodes.

The liquid crystal element may include the liquid crystal layer, a pair of substrates for retaining the liquid crystal layer, and the electrodes that are disposed on at least one of the substrates to apply voltage to the liquid crystal layer. The liquid crystal element may further include a black plate, an antireflection film, a brightness enhancing film, or the like, as necessary.

Glass, plastic, or the like may be employed as a material of the substrate constituting the liquid crystal element. Examples of the plastic employed as the substrate include an acrylic resin, a polycarbonate resin, an epoxy resin, a polyester resin, a polyamide resin, a polyolefin resin, a polyether resin, a polysulfide resin, a polysulfone resin, a polyestersulfone resin, a polyetherimide resin, a polyimide resin, and the like.

At least one of the pair of substrates constituting the liquid crystal element may be transmissive. In the case where the substrate is transmissive, its haze value may be, for example, 3% or less, preferably 2% or less, or 1% or less. The total transmittance of the transmissive substrate may be, for example, 70% or more, preferably 80% or more, or 90% or more.

A substrate may be non-transmissive. In the case where a non-transmissive substrate is employed as the substrate, a non-reflective black substrate may be employed for the side opposite to a display surface. Examples of the black substrate include a plastic substrate containing an inorganic pigment such as carbon black added thereto.

It is sufficient that the electrodes be disposed in such a manner that allows voltage application to the liquid crystal layer. The pair of electrodes may be respectively disposed on the pair of substrates to sandwich the liquid crystal layer, or disposed on one of the substrates.

The electrodes may be transparent or non-transparent. An electrode provided on the transmissive substrate may be a transparent electrode. Examples of a material constituting the transparent electrode include indium oxide, indium tin oxide (ITO), tin oxide, PEDOT-PSS, silver nanorods, carbon nanotubes, and the like. The transparent electrode can be formed by a sputtering method, a sol-gel method, or a printing method.

An electrode layer used for the substrate serving as a counterpart of the substrate having the transparent electrode out of the pair of substrates may be formed of a transparent electrode or a non-transparent electrode. Examples of the non-transparent electrode include a GC electrode and the like.

The surface of the electrode layer of the liquid crystal element may be subjected to a rubbing treatment as necessary. The rubbing treatment facilitates alignment of the liquid crystals.

In the liquid crystal element, the liquid crystal layer can be formed by giving the liquid crystal composition to a space (cell gap) formed between the pair of substrates disposed with a spacer or the like interposed therebetween. Alternatively, the liquid crystal layer can be disposed in the space between the substrates by application of the liquid crystal composition onto the substrates or by printing the substrates with the liquid crystal composition.

The liquid crystal element may include another member such as a barrier film, an ultraviolet absorbing layer, an antireflection layer, a hard coat layer, an antifouling layer, an organic interlayer insulating film, a metal reflecting plate, a phase difference plate, an alignment film, or the like. These may be used alone or in combination of two or more thereof.

The liquid crystal element can be driven by the simple matrix driving system or by the active matrix driving system in which a thin film transistor (TFT) or the like is used.

In the liquid crystal element, the absolute value of the driving voltage may be, for example, 0.1 V to 20 V, preferably 0.3 V to 15 V, or 0.5 V to 1.0 V.

Next, an example of a toning method in the liquid crystal element will be described. The liquid crystal composition containing the aromatic amine compound represented by the formula (1) as a chiral dopant, the supporting electrolyte, and the host liquid crystals is injected to a counter electrode cell. The counter electrode cell with the liquid crystal composition injected thereto shows selective reflection. Next, a direct current voltage higher than or equal to the redox potential of the chiral dopant is applied to the counter electrode cell, thereby performing toning. The variation width of the selectively reflected wavelength can be controlled by changing the molecular structure of the chiral dopant, changing the electron state, changing the application time (controlling the amount of the chiral dopant to be reacted), etc.

For returning the selectively reflected wavelength to the original, a reverse voltage needs to be applied. In a situation where the selectively reflected wavelength is changed by application of a voltage of 1.5 V, for example, the selectively reflected wavelength can be returned to the original by application of a voltage of −1.5 V. The selectively reflected wavelength of the liquid crystal composition can be changed in this manner, thereby toning light to be reflected by the liquid crystal element.

### Display Apparatus

The display apparatus includes the above-described liquid crystal element. Due to the liquid crystal element capable of toning in response to the voltage applied to the liquid crystal layer, the display apparatus of the reflective type driven by the simple matrix driving system or the active matrix driving system can be formed.

### Light Control Apparatus

The light control apparatus includes the above-described liquid crystal element. Due to the liquid crystal element capable of toning in response to the voltage applied to the liquid crystal layer, the light control apparatus that produces colors of reflected light or transmitted light having a desired circular polarization can be formed.

The present disclosure may include the following aspects.
[1] An aromatic amine compound represented by formula (1) below.

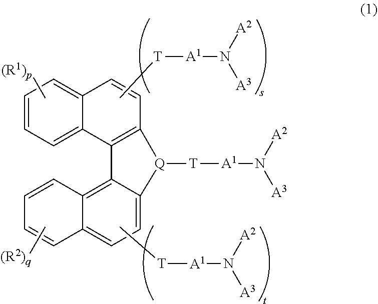

In the formula (1), A¹s each independently represent a substituted or unsubstituted alkylene group or a substituted or unsubstituted divalent aromatic group, and A²s and A³s each independently represent a substituted or unsubstituted alkyl group or a substituted or unsubstituted aromatic group, where at least one of A¹, A² and A³ represents an aromatic group. S and t each independently represent an integer from 0 to 6. R¹ and R² each independently represent a substituent. p+s and q+t each independently represent an integer from 0 to 6. Ts each independently represent a divalent linking group formed from at least one kind selected from a carbonyl group, an oxygen atom, an imino group, and an alkylene group. Q represents a trivalent linking group composed of at least one kind selected from an oxygen atom, a nitrogen atom, a carbon atom, a phosphorus atom, a sulfur atom, and a hydrogen atom.
[2] The aromatic amine compound according to [1], wherein Ts in the formula (1) are each a divalent linking group including at least a carbonyl group.
[3] The aromatic amine compound according to [1] or [2], wherein Q in the formula (1) is represented by formula (2a) or (2b) below.

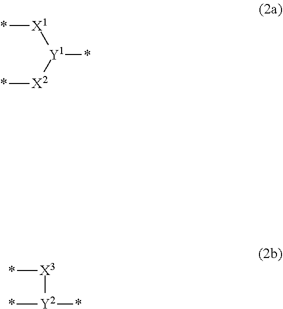

In the formulas (2a) and (2b), each * denotes a site of bonding to another atom. X¹, X² and X³ each independently include at least one kind selected from an oxygen atom, a sulfur atom, —C(R³)(R⁴)—, and —N(R⁵)—, where R³, R⁴ and R⁵ each independently represent a hydrogen atom, a substituted or unsubstituted alkyl group, or a substituted or unsubstituted aromatic group. Y¹ and Y² each independently represent one kind selected from the group consisting of a substituted or unsubstituted alkanetriyl group, a nitrogen atom, and —P(═O)(O—)—.
[4] The aromatic amine compound according to [3], wherein Q in the formula (1) is represented by the formula (2a), where X¹ and X² represent an oxygen atom.
[5] The aromatic amine compound according to [3] or [4], wherein Q in the formula (1) is represented by the formula (2a), where Y¹ represents a propane-1, 2, 3-triyl group.
[6] The aromatic amine compound according to any one of [1] to [5], wherein Ts in the formula (1) each independently represent a carbonyloxy group or an oxycarbonyl group.
[7] A liquid crystal composition containing the aromatic amine compound according to any one of [1] to [6].
[8] The liquid crystal composition according to [7], further comprising a liquid crystal compound and an electrolyte.
[9] A liquid crystal element comprising: a liquid crystal layer containing the liquid crystal composition according to [7] or [8]; and a pair of electrodes configured to apply voltage to the liquid crystal layer.
[10] A display apparatus or light control apparatus comprising the liquid crystal element according to [9].

The present disclosure includes, as another aspect, a use of the aromatic amine compound represented by the formula (1) in production of the liquid crystal composition containing the aromatic amine compound, a use of the aromatic amine compound represented by the formula (1) in production of the liquid crystal element containing the liquid crystal composition, and a use of the aromatic amine compound represented by the formula (1) in production of the liquid crystal display apparatus or light control apparatus including the liquid crystal element. The present disclosure further includes, as another aspect, the aromatic amine compound represented by the formula (1) used for the liquid crystal composition containing the aromatic amine compound, and the aromatic amine compound represented by the formula (1) used for the liquid crystal element containing the liquid crystal composition, the liquid crystal display apparatus, or the light control apparatus.

### EXAMPLES

Although the present disclosure will be specifically described below by way of Examples, the present disclosure is not limited to the Examples.

### Reference Example 1

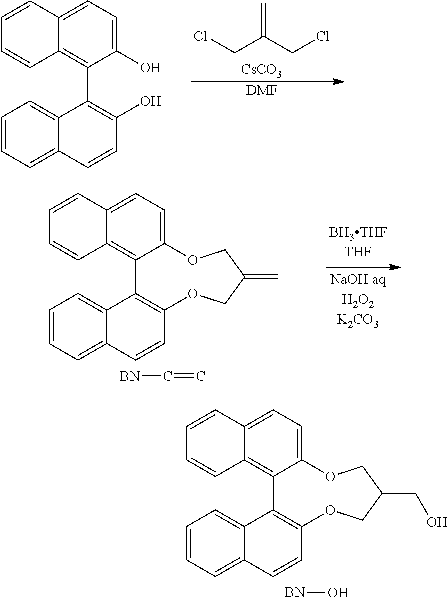

As described in the above scheme, BN—OH was synthesized with reference to a publicly known method (for example, J. Am. Chem. Soc., 2018, 140, 10946.).

### Example 1

### Synthesis of Precursor TPA-OMe-COOH

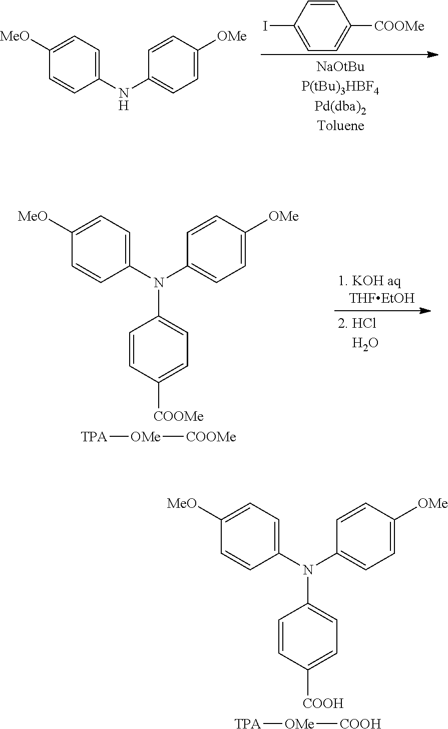

As described in the above scheme, under a nitrogen atmosphere, 1.15 g (5.0 mmol) of 4,4′-dimethoxydiphenylamine (produced by Tokyo Chemical Industry Co,. Ltd.), 1.40 g (5.2 mmol) of methyl 4-iodobenzoate (produced by Tokyo Chemical Industry Co,. Ltd.), 0.72 g (7.5 mmol) of sodium tert-butoxide (produced by Tokyo Chemical Industry Co,. Ltd.), 0.15 g (0.5 mmol) of tri-tert-butylphosphonium tetrafluoroborate (produced by Tokyo Chemical Industry Co,. Ltd.), 0.15 g (0.25 mmol) of bis(dibenzylideneacetone) palladium (0) (produced by FUJIFILM Wako Pure Chemical Corporation), and 100 mL of toluene (super dehydrated) (produced by FUJIFILM Wako Pure Chemical Corporation) were added to a three-necked flask and stirred for 7 hours under heating and refluxing, and thereafter stirred under a room temperature condition. One day after, the reaction solution was slowly added to 250 mL of a 1M aqueous ammonia solution so that the reaction was stopped. Subsequently, the solution was subjected to celite filtration, and washed with toluene. The aqueous layer was removed by separation, and furthermore, after the organic layer was subjected to separation with a saturated saline solution and dehydrated with magnesium sulfate, the solvent was removed with an evaporator, followed by vacuum drying, thereby obtaining a reddish-brown oil-like substance: TPA-OMe-COOMe.

The oil-like TPA-OMe-COOMe was dissolved in 50 mL of THF (produced by FUJIFILM Wako Pure Chemical Corporation) and 50 mL of ethanol (produced by FUJIFILM Wako Pure Chemical Corporation), and 50 mL of a 2M potassium hydroxide solution was added thereto, followed by heating and refluxing for 1 hour. After cooling, THF and ethanol were removed with an evaporator, 100 mL of ultrapure water was added thereto, and a 2M aqueous HCl solution was slowly added until the solution was acidified, thereby producing yellowish-white precipitation. Dichloromethane (produced by FUJIFILM Wako Pure Chemical Corporation) was added thereto in an amount of 200 mL to dissolve the yellowish-white precipitation, and the aqueous layer was removed by a separation operation. Subsequently, the resultant was subjected to separation with a saturated saline solution, and after the organic layer was dehydrated with magnesium sulfate, the solvent was removed with an evaporator, followed by vacuum drying, thereby obtaining a yellowish-brown oil-like substance. The oil-like substance was subjected to silica gel chromatography in which a mixture of ethyl acetate and hexane (1:1) was employed as a developing solvent, thereby isolating a target compound (Rf=0.5). After the solvent was removed with an evaporator, followed by vacuum drying, thereby obtaining 400 mg of a precursor TPA-OMe-COOH as white powder. The compound obtained was identified by ¹H-NMR.

¹H-NMR (400 MHZ, CDCl₃): δ (ppm) 7.84 (d, 2H), 7.11 (d, 4H), 6.88 (d, 4H), 6.81 (d, 2H), 3.82 (s, 6H).

### Synthesis of Compound BN-TPA-OMe

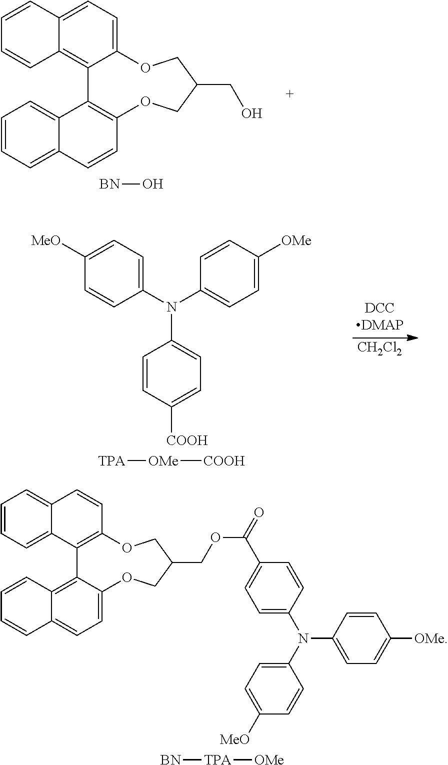

Under a nitrogen atmosphere, 0.18 g (0.5 mmol) of the compound TPA-OMe-COOH, 0.18 g (0.5 mmol) of BN—OH, 0.14 g (0.75 mmol) of 1-(3-dimethylaminopropyl)-3-ethylcarbodiimide hydrochloride (produced by Tokyo Chemical Industry Co,. Ltd.), 0.06 g (0.5 mmol) of 4-dimethylaminopyridine (produced by Tokyo Chemical Industry Co,. Ltd.), and 30 mL of dichloromethane (produced by FUJIFILM Wako Pure Chemical Corporation) were added to a three-necked flask, and the mixture was stirred for one day at room temperature. Dichloromethane was added to the reaction solution for extraction, and after separation with a saturated saline solution, the organic layer was dehydrated with magnesium sulfate. The solvent was removed with an evaporator, followed by vacuum drying, thereby obtaining pale yellow powder. A target compound BN-TPA was purified (Rf=0.5) by silica gel chromatography in which a mixture of ethyl acetate and hexane (1:1) was employed as a developing solvent. The solvent was removed with an evaporator, followed by vacuum drying, thereby obtaining 0.22 g of the final target compound BN-TPA as yellowish-white powder. Identification was made by 1H-NMR and ESI-MS.

¹H-NMR (400 MHZ, CDCl₃): δ(ppm) 7.96 (dd, 2H), 7.88 (d, 2H), 7.74 (d, 2H), 7.56 (d, 1H), 7.41 (d, 1H) 7.35-7.39 (m, 2H), 7.21-7.25 (m, 2H+2H), 7.10 (d, 4H), 6.87 (d, 4H), 6.79 (d, 2H), 4.73 (dd, 1H), 4.61 (d, 1H), 4.10-4.33 (m, 4H), 3.81 (s, 6H), 2.61-2.66 (m, 1H);

ESI-MS: m/z calc for C₄₅H₃₇NO₆: 688.27 [M+H]⁺; found 688.27.

### Example 2

### Synthesis of Precursor TPA-OC6-COOH

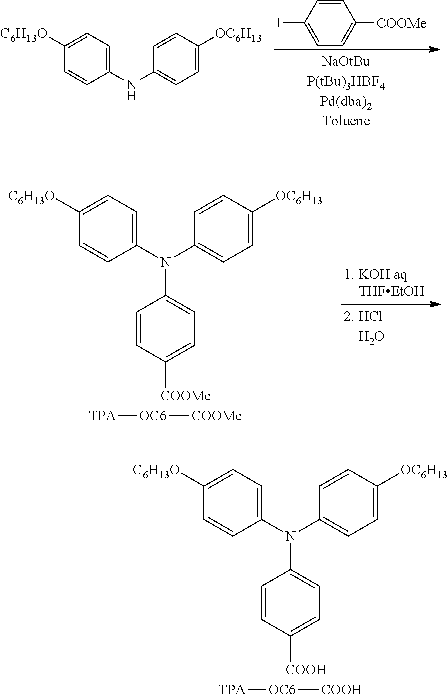

As shown in the above scheme, synthesis was performed similarly to the synthesis of the precursor TPA-OMe-COOH except that bis [4-(hexyloxy)phenyl]amine (produced by Tokyo Chemical Industry Co,. Ltd.) and methyl 4-bromobenzoate (produced by Tokyo Chemical Industry Co,. Ltd.) were employed in place of 4,4′-dimethoxydiphenylamine and methyl 4-iodobenzoate, respectively, which were employed in the synthesis of the precursor TPA-OMe-COOH. A precursor TPA-OC6-COOH obtained was identified by 1H-NMR.

¹H-NMR (400 MHZ, CDCl₃): δ(ppm) 7.83 (d, 2H), 7.10 (d, 4H), 6.86 (d, 4H), 6.80 (d, 2H), 3.93 (t, 4H), 1.74-1.81 (m, 4H), 1.42-1.50 (m, 4H), 1.30-1.40 (m, 4H+4H), 0.91 (t, 6H).

### Synthesis of Compound BN-TPA-OC6

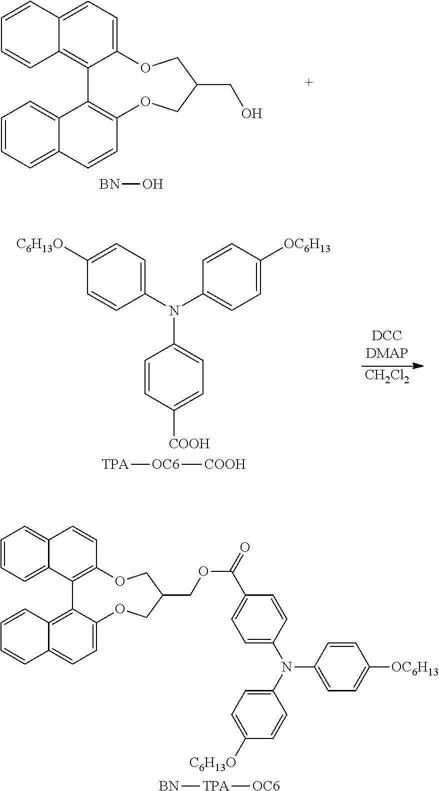

As shown in the above scheme, a compound BN-TPA-OC6 was synthesized similarly to the synthesis of the compound BN-TPA-OMe except that the precursor TPA-OC6-COOH was employed in place of the precursor TPA-OMe-COOH employed in the synthesis of the compound BN-TPA-OMe. Identification was made by 1H-NMR and ESI-MS.

¹H-NMR (400 MHZ, CDCl₃): δ(ppm) 7.96 (dd, 2H), 7.88 (d, 2H), 7.71 (d, 2H), 7.55 (d, 1H), 7.41 (d, 1H) 7.33-7.40 (m, 2H), 7.23-7.26 (m, 2H+2H), 7.08 (d, 4H), 6.84 (d, 4H), 6.79 (d, 2H), 4.73 (dd, 1H), 4.61 (d, 1H), 4.10-4.33 (m, 4H), 3.93 (s, 4H), 2.62-2.66 (m, 1H), 1.74-1.81 (m, 4H), 1.42-1.48 (m, 4H), 1.40-1.31 (m, 4H+4H), 0.91 (t, 6H);

ESI-MS: m/z calc for C₅₅H₅₇NO₆: 828.43 [M+H]⁺; found 828.43.

### Example 3

### Synthesis of Precursor TPA-Me-COOH

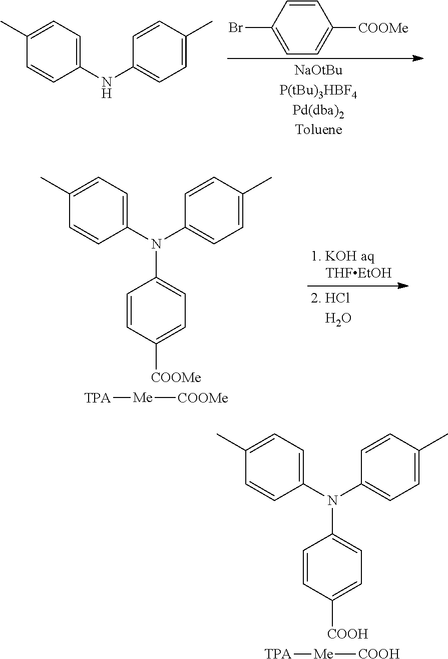

As shown in the above scheme, a precursor TPA-Me-COOH was synthesized similarly to the synthesis of the precursor TPA-OMe-COOH except that p, p′-ditolylamine (produced by Tokyo Chemical Industry Co,. Ltd.) and methyl 4-bromobenzoate (produced by Tokyo Chemical Industry Co,. Ltd.) were employed in place of 4,4′-dimethoxydiphenylamine and methyl 4-iodobenzoate, respectively, which were employed in the synthesis of the precursor TPA-OMe-COOH. Identification was made by ¹H-NMR.

¹H-NMR (400 MHZ, CDCl₃): δ(ppm) 7.86 (d, 2H), 7.13 (d, 4H), 7.05 (d, 4H), 6.91 (d, 2H), 2.34 (s, 6H).

### Synthesis of Compound BN-TPA-Me

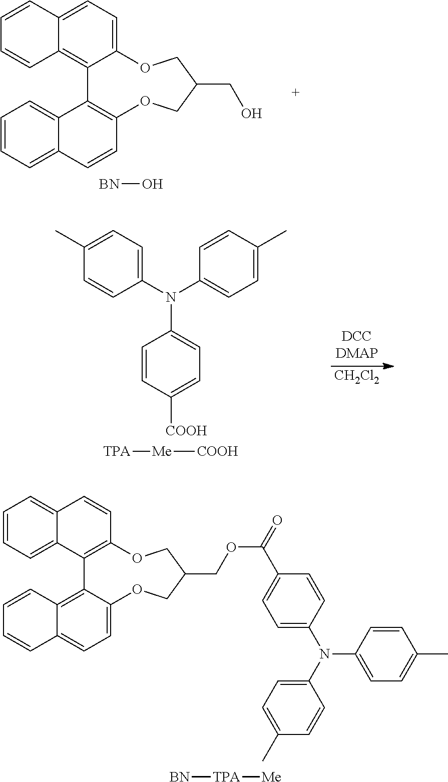

As shown in the above scheme, a compound BN-TPA-Me was synthesized similarly to the synthesis of the compound BN-TPA-OMe except that the precursor TPA-Me-COOH was employed in place of the precursor TPA-OMe-COOH employed in the synthesis of the compound BN-TPA-OMe. Identification was made by 1H-NMR and ESI-MS.

¹H-NMR (400 MHZ, CDCl₃): δ(ppm) 7.96 (dd, 2H), 7.88 (d, 2H), 7.75 (d, 2H), 7.56 (d, 1H), 7.41 (d, 1H) 7.35-7.39 (m, 2H), 7.23-7.26 (m, 2H+2H), 7.12 (d, 4H), 7.03 (d, 4H), 6.89 (d, 2H), 4.73 (dd, 1H), 4.61 (d, 1H), 4.10-4.31 (m, 4H), 2.61-2.66 (m, 1H), 2.33 (s, 6H);

ESI-MS: m/z calc for C₄₅H₃₇NO₄: 656.28 [M+H]⁺; found 656.28.

### Example 4

### Synthesis of Precursor TPA-tBu-COOH

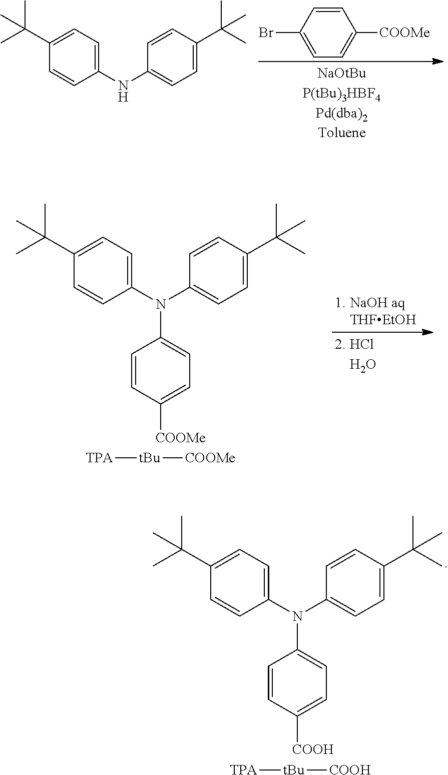

As shown in the above scheme, a precursor TPA-tBu-COOH was synthesized similarly to the synthesis of the compound TPA-OMe-COOH except that bis(4-tert-butylphenyl) amine (produced by Tokyo Chemical Industry Co,. Ltd.) and methyl 4-bromobenzoate (produced by Tokyo Chemical Industry Co,. Ltd.) were employed in place of 4,4′-dimethoxydiphenylamine and methyl 4-iodobenzoate, respectively, which were employed in the synthesis of the precursor TPA-OMe-COOH. Identification was made by 1H-NMR.

¹H-NMR (400 MHZ, CDCl₃): δ(ppm) 7.87 (d, 2H), 7.32 (d, 4H), 7.08 (d, 4H), 6.93 (d, 2H), 1.32 (s, 18H).

### Synthesis of Compound BN-TPA-tBu

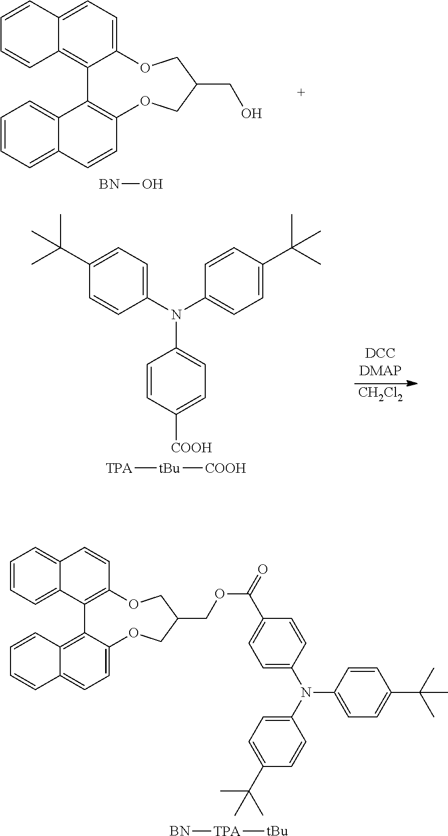

As shown in the above scheme, a compound BN-TPA-tBu was synthesized similarly to the synthesis of the compound BN-TPA-OMe except that the precursor TPA-tBu-COOH was employed in place of the precursor TPA-OMe-COOH employed in the synthesis of the compound BN-TPA-OMe. Identification was made by ¹H-NMR and ESI-MS.

¹H-NMR (400 MHZ, CDCl₃): δ(ppm) 7.96 (dd, 2H), 7.88 (d, 2H), 7.76 (d, 2H), 7.56 (d, 1H), 7.41 (d, 1H) 7.35-7.39 (m, 2H), 7.31 (d, 4H), 7.21-7.25 (m, 2H+2H), 7.06 (d, 4H), 6.91 (d, 2H), 4.73 (dd, 1H), 4.61 (d, 1H), 4.11-4.31 (m, 4H), 2.61-2.67 (m, 1H), 1.32 (s, 18H);

ESI-MS: m/z calc for C₅₁H₄₉NO₄: 740.37 [M+H]⁺; found 740.37.

### Example 5

### Synthesis of Precursor N2-H

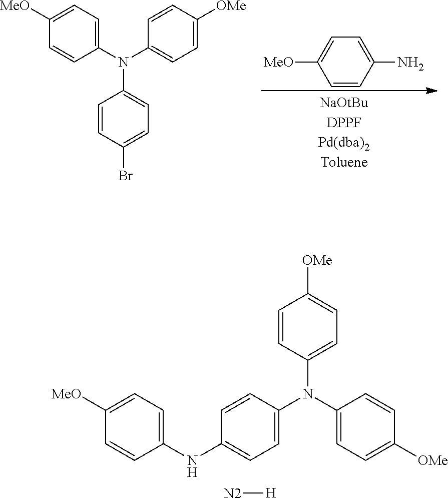

As shown in the above scheme, under a nitrogen atmosphere, 1.18 g (3.0 mmol) of 4-bromo-4,4′-dimethoxytriphenylamine (produced by Tokyo Chemical Industry Co,. Ltd.), 0.40 g (3.2 mmol) of p-anisidine (produced by Tokyo Chemical Industry Co,. Ltd.), 0.43 g (4.5 mmol) of sodium tert-butoxide (produced by Tokyo Chemical Industry Co,. Ltd.), 0.17 g (0.3 mmol) of 1,1′-bis(diphenylphosphino) ferrocene (produced by Tokyo Chemical Industry Co,. Ltd.), 0.09 g (0.15 mmol) of bis(dibenzylideneacetone) palladium (0) (produced by FUJIFILM Wako Pure Chemical Corporation), and 30 mL of toluene (super dehydrated) (produced by FUJIFILM Wako Pure Chemical Corporation) were added to a three-necked flask and stirred for 7 hours under heating and refluxing, and thereafter stirred under a room temperature condition. One day after, the reaction was stopped, and the solution was subjected to celite filtration and washed with toluene. After the organic layer was subjected to separation with a saturated saline solution and dehydrated with magnesium sulfate, the solvent was removed with an evaporator, followed by vacuum drying, thereby obtaining a reddish-brown oil-like substance. The oil-like substance was subjected to silica gel chromatography in which a mixture of ethyl acetate and hexane (1:3) was employed as a developing solvent, thereby isolating a target compound (Rf=0.4). After the solvent was removed with an evaporator, followed by vacuum drying, thereby obtaining 0.96 g of a precursor N2-H as a target yellowish-brown oil-like substance. Identification was made by ¹H-NMR.

¹H-NMR (400 MHZ, DMSO): δ(ppm) 7.71 (s, 1H), 7.00 (d, 2H), 6.80-6.89 (m, 14H), 3.74 (s, 6H), 3.71 (s, 3H).

### Synthesis of Precursor N2-COOH

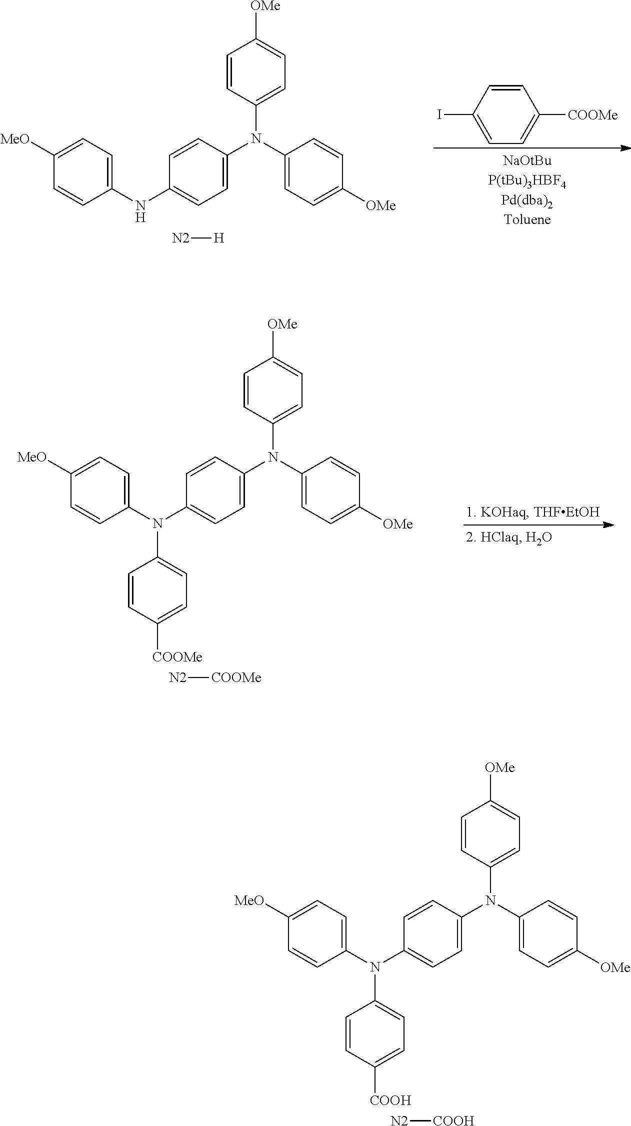

As shown in the above scheme, under a nitrogen atmosphere, 0.96 g (2.3 mmol) of N2-H synthesized, 1.1 g (4.2 mmol) of methyl 4-iodobenzoate (produced by Tokyo Chemical Industry Co,. Ltd.), 0.40 g (4.2 mmol) of sodium tert-butoxide (produced by Tokyo Chemical Industry Co,. Ltd.), 0.10 g (0.35 mmol) of tri-tert-butylphosphonium tetrafluoroborate (produced by Tokyo Chemical Industry Co,. Ltd.), 0.10 g (0.35 mmol) of bis(dibenzylideneacetone) palladium (0) (produced by FUJIFILM Wako Pure Chemical Corporation), and 50 mL of toluene (super dehydrated) (produced by FUJIFILM Wako Pure Chemical Corporation) were added to a three-necked flask and stirred for 7 hours under heating and refluxing, and thereafter stirred under a room temperature condition. One day after, the reaction solution was slowly added to 250 mL of a 1M aqueous ammonia solution so that the reaction was stopped. Subsequently, the solution was subjected to celite filtration, and washed with toluene. The aqueous layer was removed by separation, and furthermore, after the organic layer was subjected to separation with a saturated saline solution and dehydrated with magnesium sulfate, the solvent was removed with an evaporator, followed by vacuum drying, thereby obtaining a reddish-brown oil-like substance: N2-COOMe.

The oil-like N2-COOMe was dissolved in 50 mL of THE (produced by FUJIFILM Wako Pure Chemical Corporation) and 50 mL of ethanol (produced by FUJIFILM Wako Pure Chemical Corporation), and 50 mL of a 2M potassium hydroxide solution was added thereto, followed by heating and refluxing for 1 hour. After cooling, THE and ethanol were removed with an evaporator, 100 mL of ultrapure water was added thereto, and a 2M aqueous HCl solution was slowly added until the solution was acidified. Dichloromethane (produced by FUJIFILM Wako Pure Chemical Corporation) was added thereto in an amount of 200 mL, and the aqueous layer was removed by a separation operation. Subsequently, the resultant was subjected to separation with a saturated saline solution, and after the organic layer was dehydrated with magnesium sulfate, the solvent was removed with an evaporator, followed by vacuum drying, thereby obtaining a yellowish-brown oil-like substance. The oil-like substance was subjected to silica gel chromatography in which a mixture of ethyl acetate and hexane (1:1) was employed as a developing solvent, thereby isolating a target compound (Rf=0.35). After the solvent was removed with an evaporator, followed by vacuum drying, thereby obtaining 0.19 g of a precursor N2-COOH as yellowish-white powder. Identification was made by ¹H-NMR.

¹H-NMR (400 MHZ, DMSO): δ (ppm) 12.31 (s, 1H), 7.71 (d, 2H), 7.14 (d, 2H), 7.10 (d, 4H), 7.04 (d, 2H), 6.98 (d, 2H), 6.92 (d, 4H), 6.78 (d, 2H), 6.70 (d, 2H), 3.77 (s, 3H), 3.74 (s, 6H).

### Synthesis of Compound BN-N2

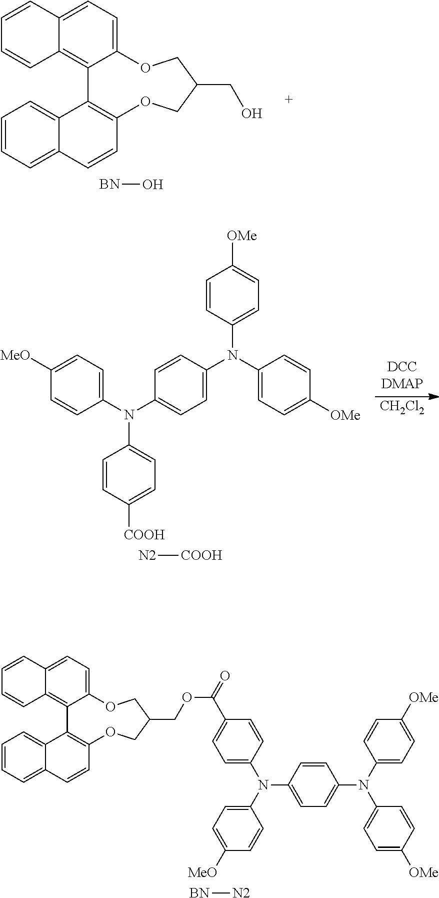

As shown in the above scheme, a compound BN—N2 was synthesized similarly to the synthesis of the compound BN-TPA-OMe except that the precursor N2-COOH was employed in place of the precursor TPA-OMe-COOH employed in the synthesis of the compound BN-TPA-OMe. Identification was made by ¹H-NMR and ESI-MS.

¹H-NMR (400 MHZ, CDCl₃): δ(ppm) 7.96 (dd, 2H), 7.88 (d, 2H), 7.74 (d, 2H), 7.56 (d, 1H), 7.41 (d, 1H) 7.33-7.39 (m, 2H), 7.21-7.25 (m, 2H+2H), 7.11 (d, 2H), 7.05 (d, 4H), 6.94 (d, 2H), 6.81-6.89 (m, 10H), 4.73 (dd, 1H), 4.61 (d, 1H), 4.10-4.30 (m, 4H), 3.81 (s, 3H), 3.78 (s, 6H), 2.60-2.65 (m, 1H);

ESI-MS: m/z calc for C₅₈H₄₈N₂O₇: 885.3534 [M+H]⁺; found 885.3477.

### Example 6

### Synthesis of Precursor N3-H

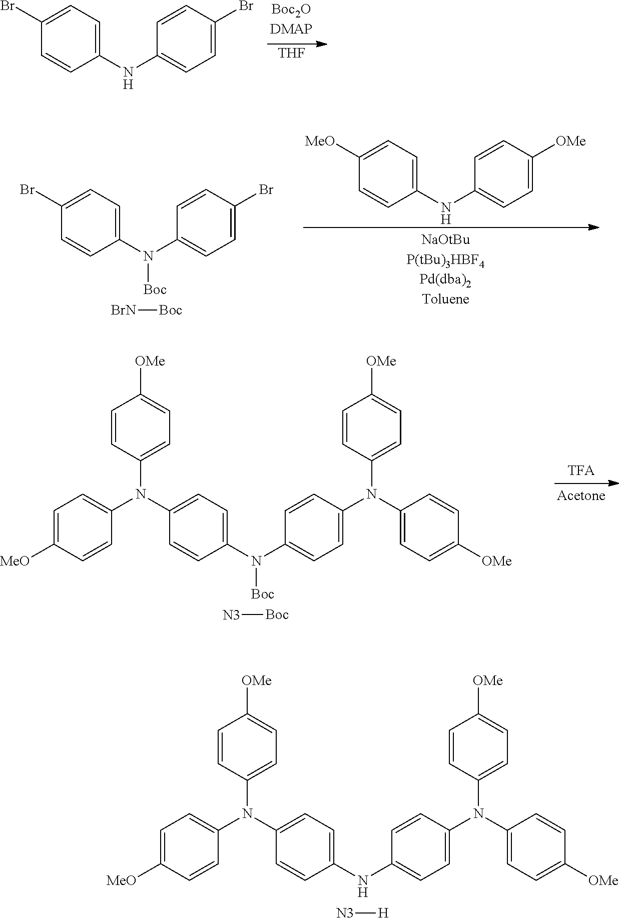

As shown in the above scheme, a precursor N3-H was synthesized with reference to a publicly known method (J. Mater. Chem. C. 2018, 6, 6429.).

### Synthesis of Precursor N3-COOH

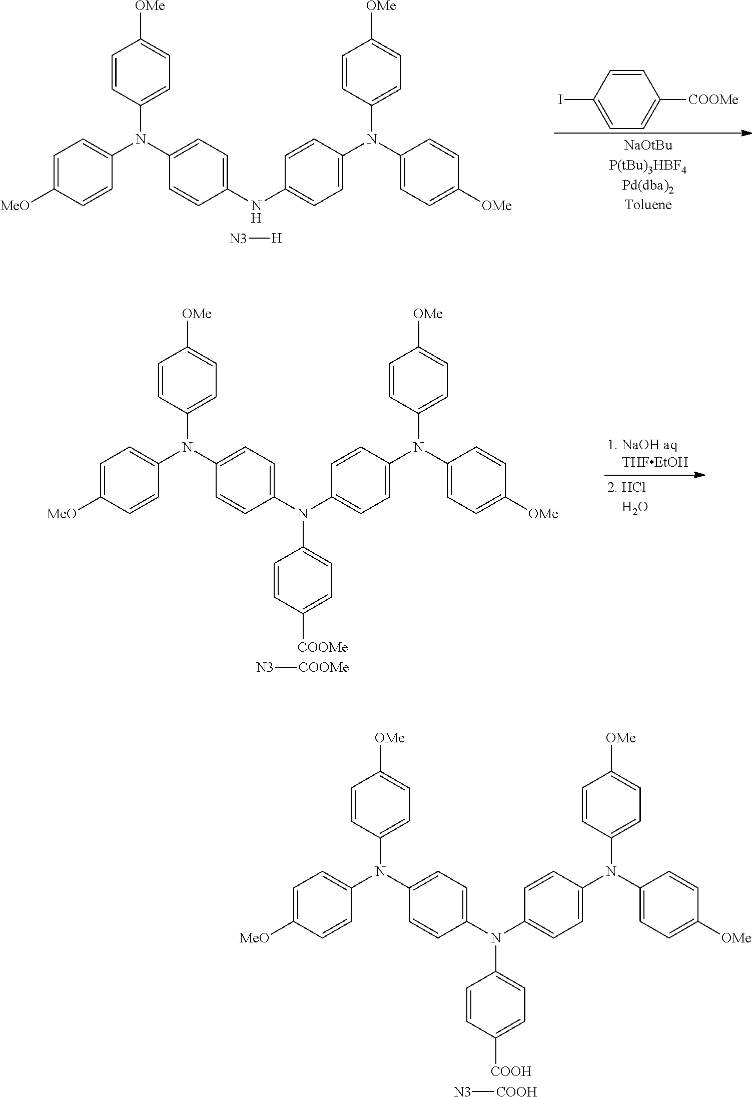

As shown in the above scheme, under a nitrogen atmosphere, 2.5 g (4 mmol) of N3-H synthesized, 1.1 g (4 mmol) of methyl 4-iodobenzoate (produced by Tokyo Chemical Industry Co,. Ltd.), 0.58 g (6 mmol) of sodium tert-butoxide (produced by Tokyo Chemical Industry Co,. Ltd.), 0.12 g (0.4 mmol) of tri-tert-butylphosphonium tetrafluoroborate (produced by Tokyo Chemical Industry Co,. Ltd.), 0.12 g (0.2 mmol) of bis(dibenzylideneacetone) palladium (0) (produced by FUJIFILM Wako Pure Chemical Corporation), and 100 mL of toluene (super dehydrated) (produced by FUJIFILM Wako Pure Chemical Corporation) were added to a three-necked flask and stirred for 7 hours under heating and refluxing, and thereafter stirred under a room temperature condition. One day after, the reaction solution was slowly added to 250 mL of a 1M aqueous ammonia solution so that the reaction was stopped. Subsequently, the solution was subjected to celite filtration, and washed with toluene. The aqueous layer was removed by separation, and furthermore, after the organic layer was subjected to separation with a saturated saline solution and dehydrated with magnesium sulfate, the solvent was removed with an evaporator, followed by vacuum drying, thereby obtaining a reddish-brown oil-like substance: N3-COOMe.

The oil-like N3-COOMe was dissolved in 50 mL of THE (produced by FUJIFILM Wako Pure Chemical Corporation) and 50 mL of ethanol (produced by FUJIFILM Wako Pure Chemical Corporation), and 50 mL of a 2M sodium hydroxide solution was added thereto, followed by heating and refluxing for 1 hour. After cooling, THF and ethanol were removed with an evaporator, 100 mL of ultrapure water was added thereto, and a 2M aqueous HCl solution was slowly added until the solution was acidified. Dichloromethane (produced by FUJIFILM Wako Pure Chemical Corporation) was added thereto in an amount of 200 mL, and the aqueous layer was removed by a separation operation. Subsequently, the resultant was subjected to separation with brine, and after the organic layer was dehydrated with magnesium sulfate, the solvent was removed with an evaporator, followed by vacuum drying, thereby obtaining a yellowish-brown oil-like substance. The oil-like substance was subjected to silica gel chromatography in which a mixture of ethyl acetate and hexane (1:1) was employed as a developing solvent, thereby isolating a target compound (Rf=0.35). After the solvent was removed with an evaporator, followed by vacuum drying, thereby obtaining 0.47 g of a precursor N3-COOH as yellowish-white powder. Identification was made by ¹H-NMR.

¹H-NMR (400 MHZ, CDCl₃): δ(ppm) 7.84 (d, 2H), 7.06 (d, 8H), 6.98 (d, 4H), 6.87 (d, 2H), 6.86 (d, 4H), 6.82 (d, 8H), 3.80 (s, 12H).

### Synthesis of Compound BN-N3

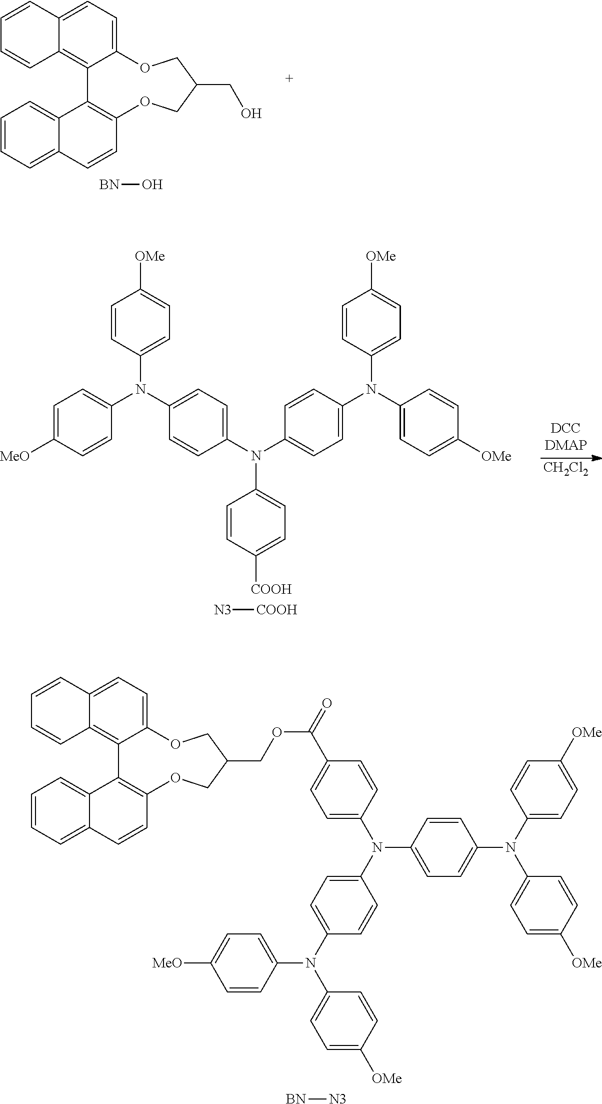

As shown in the above scheme, a compound BN—N3 was synthesized similarly to the synthesis of the compound BN-TPA-OMe except that the precursor N3-COOH was employed in place of the precursor TPA-OMe-COOH employed in the synthesis of the compound BN-TPA-OMe. Identification was made by ¹H-NMR and ESI-MS.

¹H-NMR (400 MHZ, CDCl₃): δ(ppm) 7.95 (dd, 2H), 7.87 (d, 2H), 7.75 (d, 2H), 7.55 (d, 1H), 7.41 (d, 1H) 7.33-7.39 (m, 2H), 7.21-7.25 (m, 2H+2H), 7.05 (d, 8H), 6.95 (d, 4H), 6.87 (d, 2H), 6.86 (d, 4H), 6.82 (d, 8H), 4.72 (dd, 1H), 4.60 (d, 1H), 4.09-4.32 (m, 4H), 3.78 (s, 12H), 2.60-2.65 (m, 1H);

ESI-MS: m/z calc for C₇₁H₅₉N₃O₈: 1082.4375 [M+H]⁺; found 1082.4302.

### Comparative Example 1

With reference to J. Am. Chem. Soc. 2018, 140, 10946., a comparative example compound BN-Fc represented by the following structural formula having a ferrocene skeleton in place of the aromatic amine skeleton was prepared.

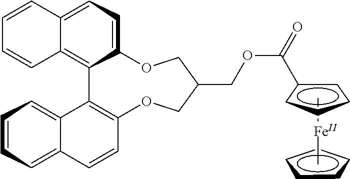

### Evaluation Electrochemical Measurement

### Preparation of Sample Solution

Each of the compounds BN-TPA-OMe, BN-TPA-OC6, BN-TPA-Me, BN-TPA-tBu, BN—N2, and BN—N3, and the comparative example compound BN-Fc prepared above was dissolved in a 100 mM tetrabutylammonium tetrafluoroborate/dichloromethane solution to give a concentration of the compound of 1 mM, thereby preparing a sample solution for electrochemical measurement.

### Measurement

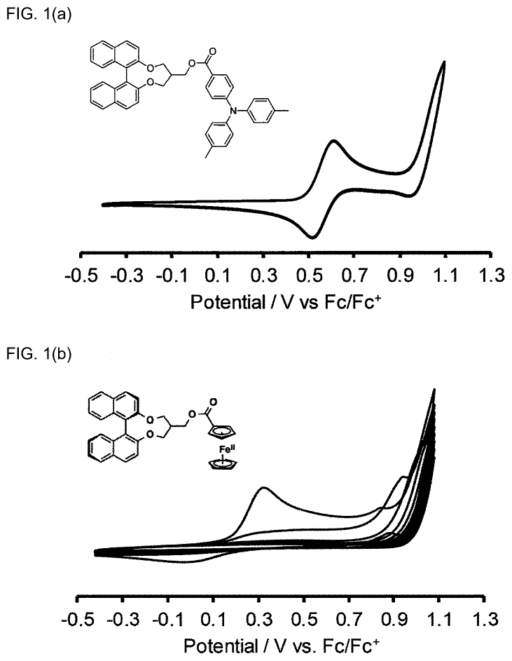

The sample solution prepared was subjected to cyclic voltammetry (CV) measurement with an electrochemical measurement device (Model 660E; produced by BAS Inc.). The measurement was performed with use of a non-aqueous Ag/Ag⁺ reference electrode RE-7 (produced by BAS Inc.) as a reference electrode, a GC electrode as a working electrode, and a platinum electrode as a counter electrode. The CV measurement was performed with a sweep voltage from −0.2 V to 1.3 V (vs. Ag/Ag⁺) and a sweep rate of 0.05 V/sec, with 10 sweeps. FIG. 1(a) shows a cyclic voltammogram of the compound BN-TPA-Me, and FIG. 1(b) shows a cyclic voltammogram of the comparative example compound BN-Fc, in which ferrocene was used as a standard. Table 1 shows redox potentials of the compounds, and results of evaluation of the compounds in terms of stability with respect to redox reactions at or above 1 V (vs. Ag/Ag⁺). Note that the stability was evaluated with reference to a waveform change in the cyclic voltammogram in the CV measurement in which 10 sweeps were carried out. Specifically, in the case where there was no change between a waveform after 1 sweep and a waveform after 10 sweeps, the compound was evaluated as “stable”.

TABLE 1

| | Redox | Redox | Redox | Stability |
|---|---|---|---|---|
| Name of | potential 1 | potential 2 | potential 3 | at or above |
| compound | [V] | [V] | [V] | 1 V |
| BN-TPA-OMe | 0.39 | — | — | Stable |
| BN-TPA-OC6 | 0.34 | — | — | Stable |
| BN-TPA-Me | 0.56 | — | — | Stable |
| BN-TPA-tBu | 0.56 | — | — | Stable |
| BN-N2 | 0.05 | 0.45 | — | Stable |
| BN-N3 | −0.01 | 0.23 | 0.72 | Stable |
| BN-Fc | 0.27 | — | — | Unstable |

As shown in FIG. 1 and Table 1, it can be seen that the compounds according to Examples were stable with respect to redox reactions. Furthermore, it can also be seen that the compounds having a plurality of aromatic amine moieties showed a plurality of redox potentials.

### Evaluation Absorption Spectrum Measurement

### Preparation of Sample Solution

Each of the compounds BN-TPA-OMe, BN-TPA-OC6, BN-TPA-Me, BN-TPA-tBu, BN—N2, and BN—N3, and the comparative example compound BN-Fc prepared above was dissolved in dichloromethane to give a concentration of the compound of 0.1 mM, thereby preparing a sample solution for absorption spectrum measurement.

### Measurement

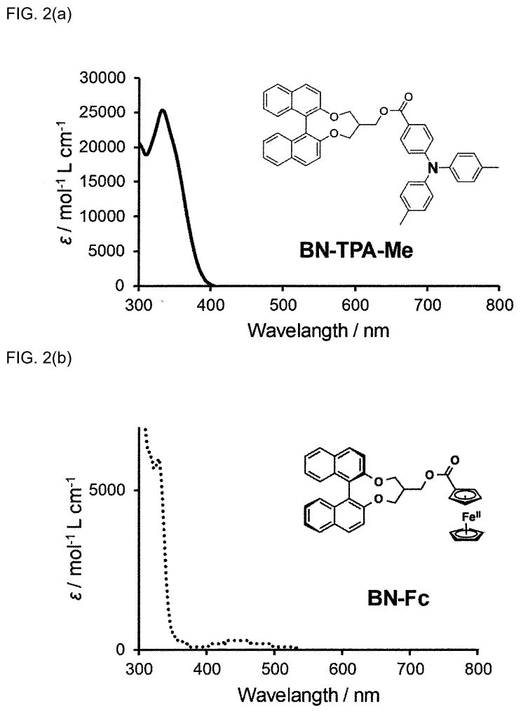

The prepared sample solution was put into a quartz cell having an optical path length of 0.1 cm, and the absorption spectrum was measured with an ultraviolet-visible spectrophotometer (UV1800 produced by Shimadzu Corporation). FIGS. 2(a) and 2(b) show absorption spectra of the compound BN-TPA-Me and the comparative example compound BN-Fc, respectively. Table 2 shows absorption edge wavelengths, and tones shown in the form of liquid crystal compositions. Note that the tones shown in the form of liquid crystal compositions were evaluated by visual observation of liquid crystal composition samples for transmission spectrum measurement described later.

TABLE 2

| | Absorption edge | Tone of liquid crystal |
|---|---|---|
| Compound | wavelength [nm] | composition sample |
| BN-TPA-OMe | 416 | Colorless |
| BN-TPA-OC6 | 423 | Colorless |
| BN-TPA-Me | 403 | Colorless |
| BN-TPA-tBu | 406 | Colorless |
| BN-Fc | 532 | Orange |

It can be seen that colorless liquid crystal compositions can be formed with use of the compounds according to Examples.

### Evaluation Measurement of Transmission Spectrum of Liquid Crystal Composition

Each of the compounds BN-TPA-OMe, BN-TPA-OC6, BN-TPA-Me, and BN-TPA-tBu, and the comparative example compound BN-Fc was dissolved in a solution of host liquid crystal molecules composed of a mixture of 4-cyano-4′-pentyloxybiphenyl and 4-cyano-4′-pentylbiphenyl (7:3) in methylene chloride to give a final concentration of 3 mol %, and after 3 mol % of 1-ethyl-3-methylimidazolium triflate was added thereto, the solution was concentrated under reduced pressure, thereby preparing a liquid crystal composition sample.

### Measurement

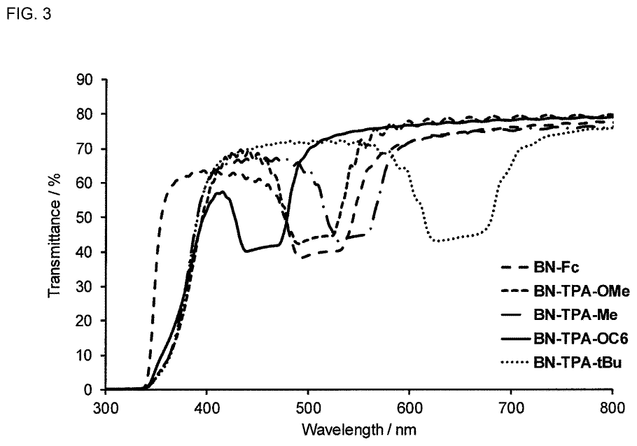

The liquid crystal composition sample prepared above was introduced into a glass cell (produced by EHC Co., Ltd.), which had a cell thickness of 5 μm and a polyimide alignment film subjected to rubbing treatment, and was caused to develop cholesteric liquid crystals at room temperature, and the transmission spectrum was measured. The results are shown in FIG. 3 and Table 3. Note that the reflection wavelength in Table 3 is represented by a median, and the reflected color is based on evaluation by visual observation.

TABLE 3

| | Reflection | |
|---|---|---|
| Compound | wavelength [nm] | Reflected color |
| BN-TPA-OC6 | 458 | Bluish purple |
| BN-TPA-OMe | 506 | Bluish green |
| BN-TPA-Me | 544 | Yellowish green |
| BN-TPA-tBu | 655 | Red |
| BN-Fc | 514 | Bluish green |

It can be seen that, by forming a liquid crystal element having cholesteric liquid crystals developed from the compounds according to Examples, various reflected colors from red to bluish purple can be developed.

### Evaluation Change in Reflected Color Due to Voltage Application

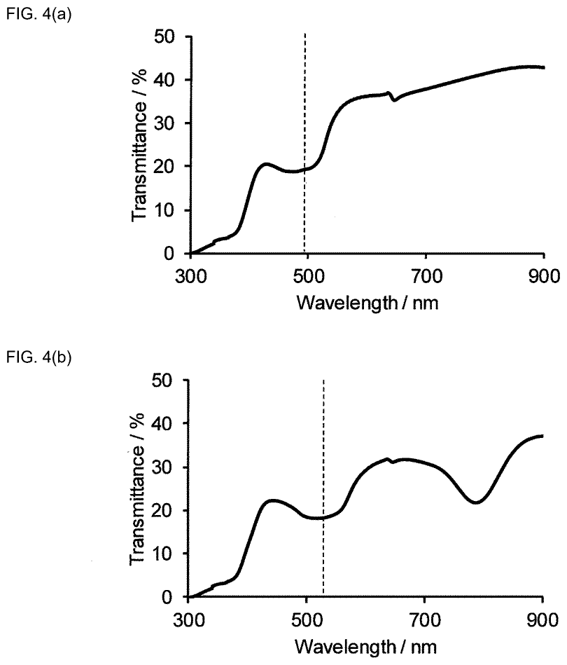

The liquid crystal composition sample prepared above was introduced into an ITO glass cell which had a cell thickness of 10 μm and produced by application of ITO glass and Prussian blue nanoparticles by spin-coating, thereby producing a liquid crystal element. Regarding the liquid crystal element produced, a change in transmission spectrum before and after application of a direct current voltage (2 V) was measured, thereby measuring a change in reflected color due to the voltage application. FIG. 4 shows a result as to a liquid crystal element containing BN-TPA-OMe as a chiral dopant, as a representative example. FIG. 4(a) shows a transmission spectrum before application of the direct current voltage (2 V), whereas FIG. 4(b) shows a transmission spectrum after application of the direct current voltage (2 V). The reflection wavelength (median) in FIG. 4(a) was 499 nm, which corresponds to bluish green, whereas the reflection wavelength (median) in FIG. 4(b) was 535 nm, which corresponds to green.

It can be seen from FIG. 4 that a liquid crystal element, the reflection wavelength of which changes in response to voltage application, can be formed from the compounds according to Examples.

The disclosure of Japanese Patent Application No. 2022-177592 (filed on Nov. 4, 2022) is incorporated herein in its entirety by reference. All the documents, patent applications, and technical standards described herein are incorporated herein by reference in their entireties to the same extent as in the case where the individual documents, patent applications, and technical standards are specifically and individually written to be incorporated by reference.

1. An aromatic amine compound represented by: 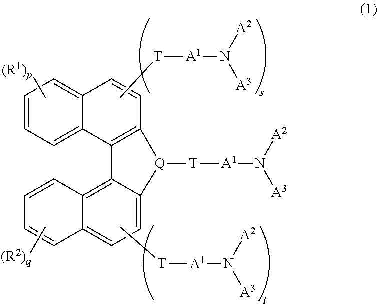
wherein: A¹s each independently represent a substituted or unsubstituted alkylene group or a substituted or unsubstituted divalent aromatic group, A²s and A³s each independently represent a substituted or unsubstituted alkyl group or a substituted or unsubstituted aromatic group, where at least one of A¹, A² and A³ represents an aromatic group, s and t each independently represent an integer from 0 to 6, R¹ and R² each independently represent a substituent, p+s and q+t each independently represent an integer from 0 to 6, Ts each independently represent a divalent linking group formed from at least one kind selected from a carbonyl group, an oxygen atom, an imino group, and an alkylene group, and Q represents a trivalent linking group composed of at least one kind selected from an oxygen atom, a nitrogen atom, a carbon atom, a phosphorus atom, a sulfur atom, and a hydrogen atom.

2. The aromatic amine compound according to claim 1, wherein Ts are each a divalent linking group including at least a carbonyl group.

3. The aromatic amine compound according to claim 1, wherein
Q is (2a) or (2b): 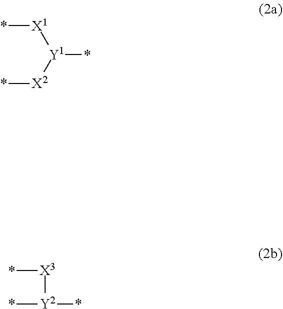
wherein each * denotes a site of bonding to another atom, X¹, X² and X³ each independently include at least one kind selected from an oxygen atom, a sulfur atom, —C(R³)(R⁴)—, and —N(R⁵)—, where R³, R⁴ and R⁵ each independently represent a hydrogen atom, a substituted or unsubstituted alkyl group, or a substituted or unsubstituted aromatic group, and Y¹ and Y² each independently represent one kind selected from a substituted or unsubstituted alkanetriyl group, a nitrogen atom, and —P(═O)(O—)—.

4. The aromatic amine compound according to claim 3, wherein Q is represented by (2a), and X¹ and X² represent an oxygen atom.

5. The aromatic amine compound according to claim 4, wherein Y¹ represents a propane-1, 2, 3-triyl group.

6. The aromatic amine compound according to claim 3, wherein Q is represented by the formula (2a), and Y¹ represents a propane-1, 2, 3-triyl group.

7. The aromatic amine compound according to claim 3, wherein Q is represented by (2b).

8. The aromatic amine compound according to claim 1, wherein Ts each independently represent a carbonyloxy group or an oxycarbonyl group.

9. A liquid crystal composition containing the aromatic amine compound according to claim 1.

10. The liquid crystal composition according to claim 9, further comprising a liquid crystal compound and an electrolyte.

11. The liquid crystal composition according to claim 9, wherein a content of the aromatic amine compound in the liquid crystal composition is 0.1 mol % to 10 mol %.

12. A liquid crystal element comprising:
a liquid crystal layer containing the liquid crystal composition according to claim 9; and
a pair of electrodes configured to apply voltage to the liquid crystal layer.

13. A display apparatus or light control apparatus comprising the liquid crystal element according to claim 12.
